(19) United States
(12) Patent Application Publication
(10) Pub. No.: US 2026/0248413 A1
(43) Pub. Date: Aug. 27, 2026

(54) SYSTEMS AND METHODS FOR PREVENTATIVE FALL RISK ASSESSMENT

(71) Applicants: The Regents of the University of Michigan, Ann Arbor, MI (US); Liberty Mutual Insurance Company, Boston, MA (US)

(72) Inventors: Jesse V. Jacobs, Northborough, MA (US); Hoonyong Lee, Zetland (AU); John Sohn, Ann Arbor, MI (US); SangHyun Lee, Ann Arbor, MI (US)

(21) Appl. No.: 19/060,415

(22) Filed: Feb. 21, 2025

(51) Int. Cl. A61B 5/11; A61B 5/00; A61B 5/0531

(52) U.S. Cl. CPC A61B 5/1117; A61B 5/0531; A61B 5/7267; A61B 5/7275; A61B 5/7282; A61B 5/742; A61B 5/746; A61B 2562/0219

(57) ABSTRACT

Methods and systems are herein provided for preventative fall risk assessment and fall risk event geolocation. In one example, a method comprises acquiring body movement data an IMU of a wearable fall risk monitoring device; acquiring biosensor data with a biosensor of the wearable fall risk monitoring device; determining, based on the body movement data and the biosensor data, a fall risk score indicating a level of fall hazard exposure experienced by a wearer of the wearable fall risk monitoring device; and in response to the fall risk score exceeding a threshold, providing a notification. Further, fall risk scores may be aggregated, geolocated, and presented in a fall risk exposure map.

### TECHNICAL FIELD

Embodiments of the subject matter disclosed herein relate to monitoring human states, and more particular to preventative fall risk assessment based on body motion and biosensor data.

### BACKGROUND

Falls are the second leading cause of unintentional fatalities worldwide. Further, non-fatal falls are the leading cause of unintentional injury leading to hospitalizations or emergency room visits, thus necessitating medical treatment, impeding physical activity, and accruing medical cost. As the incidence of fall related injuries continues to rise, fall prevention has become a focus of maintaining and improving the health and quality of people's daily lives. While traditional technological strategies for addressing falls have emphasized immediate fall detection to facilitate timely medical responses within the ‘golden hour’, such reactive approaches do not address the root causes of falls and are thus limited in their effectiveness for fall prevention.

### BRIEF DESCRIPTION

Methods and systems are provided for assessing a risk of a person falling based on body movement data and biosensor data collected via a wearable device. In one example, a method comprises acquiring body movement data with one or more motion sensors of a wearable fall risk monitoring device; acquiring biosensor data with one or more biosensors of the wearable fall risk monitoring device; determining, based on the body movement data and the biosensor data, a fall risk score indicating potential exposure to fall hazards; and in response to the fall risk score exceeding a threshold, notifying the wearer via one or more of the wearable fall risk monitoring device and a remote device communicatively coupled to the wearable fall risk monitoring device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of a fall risk assessment system, in accordance with one or more embodiments of the present disclosure;

FIG. 2 shows a block diagram of an exemplary embodiment of a training system for training a fall risk assessment model, in accordance with one or more embodiments of the present disclosure;

FIG. 3 shows a flowchart illustrating an exemplary method for training a fall risk assessment model, in accordance with one or more embodiments of the present disclosure;

FIG. 4 shows a flowchart illustrating an exemplary method for fall risk assessment, in accordance with one or more embodiments of the present disclosure;

FIG. 5 shows a flowchart illustrating an exemplary method for determining anomalous movement based on body motion data, in accordance with one or more embodiments of the present disclosure;

FIG. 6 shows a flowchart illustrating an exemplary method for determining instances of increased arousal based on biosensor data, in accordance with one or more embodiments of the present disclosure;

FIG. 7 shows a flowchart illustrating an exemplary method for geolocating fall risk events, in accordance with one or more embodiments of the present disclosure;

FIG. 8 shows a graph of angular velocities illustrating normal and abnormal body movement data, in accordance with one or more embodiments of the present disclosure;

FIG. 9A shows a graph of the angular velocities of FIG. 8 as nodes and edges, in accordance with one or more embodiments of the present disclosure;

FIG. 9B shows an enlarged section of the graph of FIG. 9A, in accordance with one or more embodiments of the present disclosure;

FIG. 10 shows graphs of biosensor data, in accordance with one or more embodiments of the present disclosure; and

FIG. 11 shows a graphical output of geolocated fall risk events, in accordance with one or more embodiments of the present disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

To reduce a risk of fall-related injuries, a fall risk assessment system is disclosed for detecting abnormal body movements based on body movement data and detecting physiological arousal based on biosensor data. The body movement data and the biosensor data are acquired by a wearable monitoring device. For example, when exposed to fall hazards while walking (e.g., unstable surfaces like wet or slippery surfaces, uneven terrain, floor openings, seasonal hazards like icy surfaces, and the like), people often adopt a corrective stepping response to recover balance. This action produces unusual bodily movements that are distinct from those observed during regular walking. However, fall risk assessment based on body movement data alone can sometimes over-detect fall risks. For example, certain bodily movements are fall-like, such as bending to pick up an object, may generate similar motion patterns to those of fall hazard exposure. Thus, assessment of fall risk based on body movement alone may detect both fall hazard exposure movements as well as fall-like body movements, decreasing the overall accuracy of the assessment.

Similar to the abnormal body movements induced by exposure to fall hazards, people's arousal levels tend to change drastically when exposed to fall hazards, typically accompanied by a peak in arousal. Such drastic changes in arousal are observed when exposed to fall hazards because stressful stimuli activate the sweat glands. In this regard, biosensor data may be acquired by the wearable monitoring device and together with the body movement data used to assess fall risk.

Abnormal body movements caused by imbalance and/or exposure to fall hazards that may be considered as a precursor to falls may be detected by a machine learning (ML) model of the fall risk assessment system, based on body movement data collected via a wearable monitoring device. Further, anomalies in physiological arousal may be identified from acquired biosensor data. The anomalies in physiological arousal and abnormal body movements may be considered together to identify fall risk exposure events. When identified, a wearer of the device may be notified of an increased risk of falling, such that the wearer may adjust their stance, posture, gait, or stride to reduce the risk of falling. Additionally, a notification may be sent to a remote device of a caregiver, family member, or other individual or entity to inform them of the increased risk of the wearer falling, which may prompt a personalized intervention. These interventions can include professional consultations, customized physical training sessions, the provision of individual feedback on walks of the wearer that may help those vulnerable to falls avoid injuries. Further, detected exposure events may be geolocated to generate a map of events. By geolocating the events, proactive interventions can be applied to the environment, or to relevant processes or behaviors to reduce fall risk before fall events occur. Use cases may include, but are not limited to, improving safety within private, commercial, or public spaces as well as identifying individual fall risk.

As opposed to other ML approaches that rely on supervised learning, where an ML model is trained to compare patterns in the body movement data with predetermined normal or typical patterns, the ML model disclosed herein is trained using unsupervised learning, where the time series body movement data is directly analyzed in context without relying on predefined regular walking patterns. Because of a high degree of variability of walking behavior, the ML model disclosed herein may allow for more accurate fall risk assessment under a wider range of walking conditions than other ML approaches. In this way, the fall risk assessment system provides a personalized and scalable solution for preventing falls in people's daily lives. Further, assessing fall risk and identifying fall risk exposure events based on both ML outputs relating to body movement data as well as physiological arousal may mitigate erroneous detection of fall-like events, thereby providing for more accurate fall risk exposure detection.

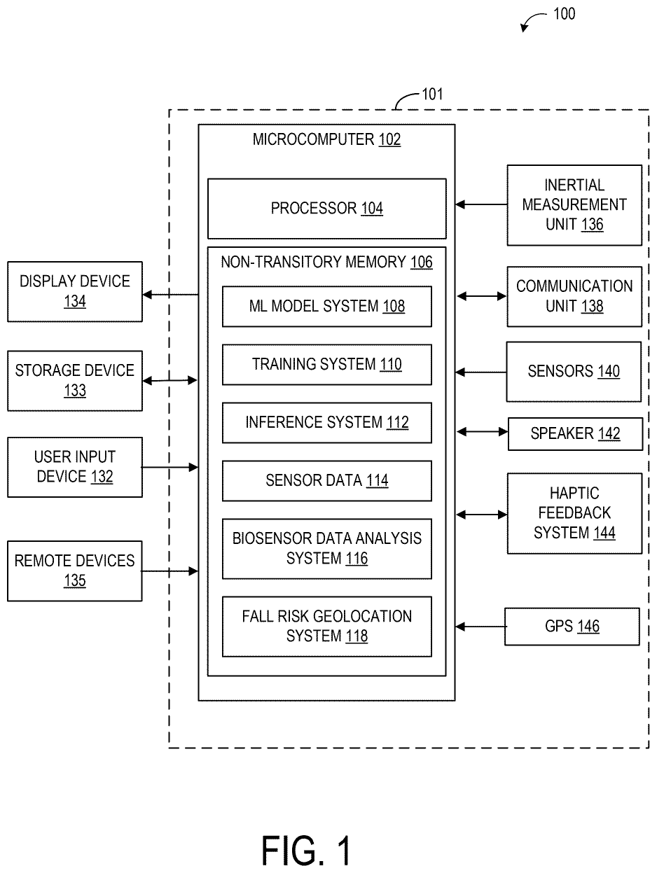

Referring to FIG. 1, a fall risk assessment system 100 is shown. In some examples, the fall risk assessment system 100 may include, at least in part, a wearable fall risk monitoring device 101. In some examples, the fall risk assessment system 100 may comprise a microcomputer 102, an inertial measurement unit (IMU) 136, one or more additional sensors 140, and a communication unit 138 included within a wearable fall risk monitoring device 101. The one or more additional sensors 140 may include one or more biosensors such as electrodermal activity (EDA) sensors configured to detect changes in electrical activity of the wearer's skin resulting from changes in sweat gland activity. The IMU 136 may comprise a plurality of motion sensors, including orthogonal accelerometers, gyroscopes, and/or magnetometers. The IMU 136 may be configured to acquire data of motion and orientation of a body or a part of a body of a wearer of the wearable fall risk monitoring device 101. In particular, the IMU 136 may acquire the motion and orientation data while the wearer is walking, running, exercising, dancing, or otherwise physically moving about an environment. In various examples, the IMU 136 and the biosensor(s) may acquire data continuously and in real-time, and transmit the data to the microcomputer 102 in real-time.

The communication unit 138 may comprise a radio communication device, such as a Bluetooth™ device, or other device capable of transmitting and/or receiving signals, including signals that include sensor data from the IMU 136 and/or the one or more additional sensors 140. The one or more additional sensors 140 may additionally comprise electrodermal activity sensors, optical sensors, electrocardiogram, light sensors, and/or the like. For example, optical sensors may be configured to determine parameters such as heart rate, blood oxygen levels, and the like based on how light reflects off the skin.

In some examples, the microcomputer 102 may be communicatively coupled to a display device 134 and a user input device 132. For example, the microcomputer 102 may be communicatively coupled to the display device 134 and the user input device 132 via the communication unit 138. The microcomputer 102 may also be communicatively coupled to one or more remote devices 135 via the communication unit 138. In various embodiments, the one or more remote devices 135 may include a computing device of a person related to or caring for the wearer. For example, the one or more remote devices 135 may include a smart phone, tablet, or personal computer (e.g., a desktop computer or laptop computer) of the wearer or a caregiver of the wearer, such as a doctor, physical therapist, or other medical professional; a family member of the wearer; an individual working at a community or residence home of the wearer; a friend of the wearer; a representative of a business or a real estate site; a workplace supervisor or manager; or a different person. As described in greater detail herein, the one or more remote devices 135 may be notified of an increased risk of falling of the wearer. In response to being notified of the increased risk, or in response to a pattern of increased risks of falling, a user of the one or more remote devices 135 may intervene with the wearer, for example, with a professional consultation, proposed physical therapy session, etc. A user or monitor of the devices may also intervene by improving the environment or processes and behaviors related to the fall risk (e.g., by fixing uneven road surface, removing hazards, etc.).

In some embodiments, at least a portion of the microcomputer 102 is communicably coupled to the IMU 136, communication unit 138, and/or one or more additional sensors 140 via wired and/or wireless connections. In some embodiments, the display device 134 and/or the user input device 132 may be disposed at a separate device than the wearable fall risk monitoring device 101. For example, the user input device 132 and the display device 134 may be included in a smart phone, tablet, workstation, desktop computer, or virtually any other similar computing device. In other examples, the wearable fall risk monitoring device 101 may comprise the display device 134 and/or the user input device 132 (e.g., via a touchscreen display). Further, although the microcomputer 102 is shown and described as being located within the wearable fall risk monitoring device 101, it should be understood that in other examples, the microcomputer 102 may be located remotely and in communication with the IMU 136 and one or more additional sensors 140 so as to analyze the acquired data at the remote location. In this situation, the fall risk monitoring device may utilize the processor 104 with reduced resources and lower power consumption. The processor's role may include collection of data from the sensors, including the IMU 136 and the one or more additional sensors 140, execution of calculations, and transmission of this information to a remote device 135.

In some embodiments, the fall risk assessment system 100 may incorporate audio feedback via a speaker 142, as well as various types of haptic feedback in a haptic feedback system 144 that is communicatively coupled to the microcomputer 102. These can include, but are not limited to, commonly used methods such as vibration, electrical stimulation, force feedback, or even thermal changes. The haptic feedback system 144, in its simplest use, may function akin to a cell phone in vibration mode, transmitting coded messages to the wearer. However, the system may take on additional importance in the context of assessing a risk of the wearer falling. Specifically, the haptic feedback system 144 may be employed to indicate an increased risk of falling to the wearer, such that the wearer may adjust their body position, stride, gait, foot placement, and/or take other precautions to reduce the risk of falling.

The microcomputer 102 may include a processor 104 configured to execute machine readable instructions stored in non-transitory memory 106. Processor 104 may be single-core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

Non-transitory memory 106 may store an ML model system 108, a model training system 110, an inference system 112, sensor data 114, a biosensor data analysis system 116, and a fall risk geolocation system 118. The ML model system 108 may include a deep learning or machine learning fall risk assessment model and instructions for implementing the fall risk assessment model to analyze data acquired by the fall risk assessment system 100, including at least portions of the sensor data 114, such as IMU data, as described in further detail below. ML model system 108 may further include various data, or metadata, pertaining to the fall risk assessment model.

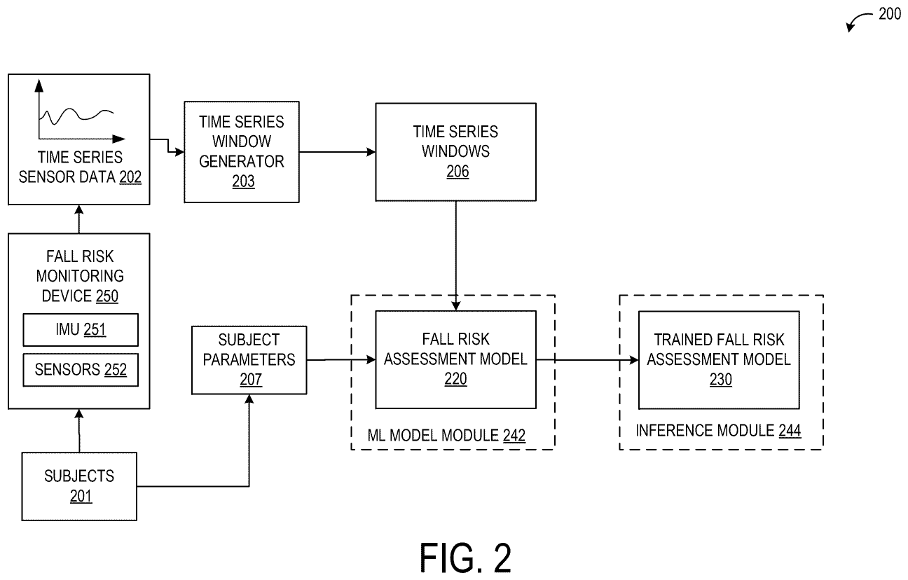

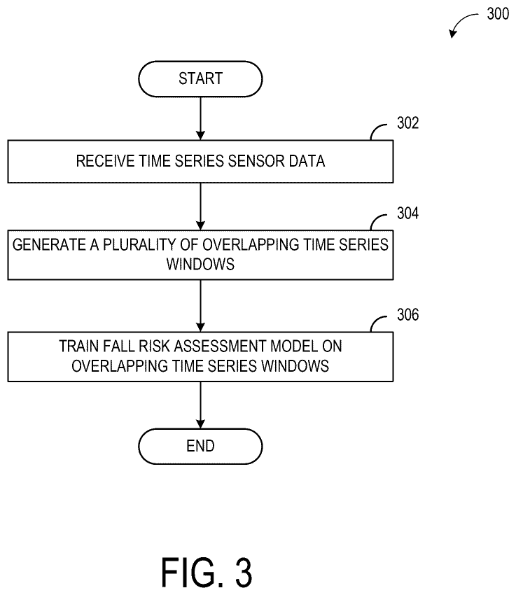

The training system 110 may comprise instructions for training the fall risk assessment model. In particular, training system 110 may include instructions that, when executed by the processor 104, cause microcomputer 102 to conduct one or more steps of method 300 for training the fall risk assessment model, discussed in more detail below with reference to FIGS. 2 and 3. Non-transitory memory 106 also stores the inference system 112 that comprises instructions for analyzing new body movement data with the trained fall risk assessment model.

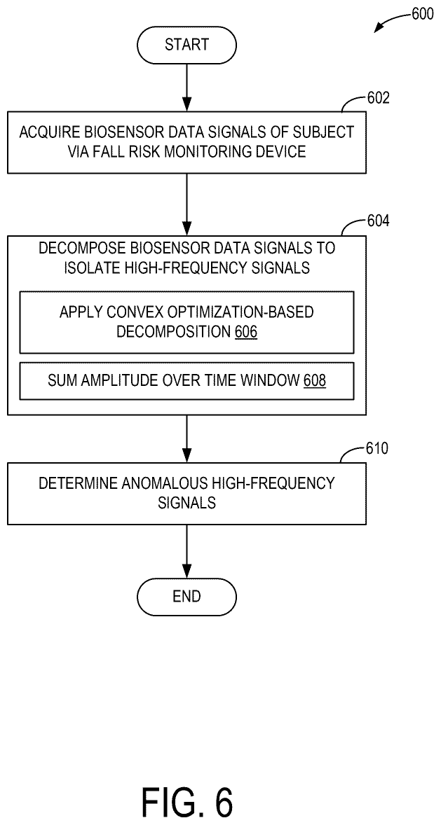

Non-transitory memory 106 further stores the biosensor data analysis system 116 that comprises instructions for identifying, based on acquired biosensor data, anomalous arousal instances. As will be further described with respect to FIG. 6, analyzing the acquired biosensor data may include signal smoother to filter out irregular fluctuations caused by electronic interference and environment noise, which may include decomposing the smoothed data signals to isolate high-frequency signals and then identifying anomalous high-frequency signals.

Non-transitory memory 106 further stores sensor data 114. Sensor data 114 may include, for example, data acquired with the IMU 136, including data acquired with the plurality of motion sensors thereof, and data acquired with the one or more biosensors. Sensor data 114 may further include environmental and/or body temperature data, optically acquired data (e.g., heart rate and/or blood oxygen data), and more, as acquired by the one or more additional sensors 140. In this way, the sensor data 114 may store data acquired from a wearer of the wearable fall risk monitoring device 101. In some examples, the stored sensor data 114 may be acquired and stored in real-time (e.g., without intentional delay). The sensor data 114 may be timestamped to allow for time aligning of data among data of different sensors. In some examples, the sensor data 114 may include acquired data over a defined time period, for example 5 minutes, where data older than the defined time period is automatically deleted from the memory 106. For example, time series body movement data acquired from the wearer may be buffered in sensor data 114 while an analysis of the time series body movement data is performed. In some embodiments, some or all of the acquired sensor data 114 may be stored in an external storage device 133. The acquired sensor data 114 may be used as inputs into the fall risk assessment model, which may analyze the sensor data 114 to detect abnormalities in a body movement of the wearer that may indicate an increased risk of falling.

In some examples, the fall risk assessment system 100 further comprises a global positioning system (GPS) 146. The GPS 146 may be configured to determine a position of the wearable device 101. The position data may be acquired in real-time in a time series. For example, the sensor data that is acquired by the IMU and the biosensor(s) may be time-aligned with the position data so as to allow for geolocation of identified fall risk exposure events. It should be understood that while the GPS 146 is depicted in FIG. 1 as being included in the wearable device 101, in other examples the GPS 146 may be disposed elsewhere (e.g., as a remote device that is communicatively coupled to the wearable device 101). The fall risk geolocation system 118 may include instructions for geolocating identified fall risk exposure events. For example, once a fall risk exposure event is identified based on the IMU data and the biosensor data, position data may be time aligned to identify a position of the wearable device at the time of the fall risk exposure event. The fall risk geolocation system 118 may additionally be configured to output the geolocated events to the display device 134, for example in a graphical display. Other location technologies like Wi-Fi, Bluetooth™, ultra-wide band, RFiD, and others may be used as alternatives to GPS, in some examples.

In some embodiments, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration.

The microcomputer 102 may be operably/communicatively coupled to the user input device 132 and the display device 134. As described previously, user input device 132 may comprise one or more of a touchscreen, a keyboard, a mouse, a trackpad, a motion sensing camera, or other device configured to enable a user to interact with and manipulate data within the microcomputer 102. Display device 134 may include one or more display devices utilizing virtually any type of technology. In examples in which the microcomputer 102 is a remote device separate from the wearable fall risk monitoring device 101, display device 134 may be combined with processor 104, non-transitory memory 106, and/or user input device 132 in a shared enclosure. For example, a smart phone may be a remote device that includes a display device and processor/memory and is communicatively coupled to the wearable fall risk monitoring device 101. As another example, the remote device may be smart phone with a display device and the wearable device may have its own display device. The types and configurations of data that are displayed on the display device of the remote device may be different from that of the data that is displayed on the wearable device, as a result of GPU power and screen size. Alternatively, display device 134 may be peripheral display devices and may comprise a monitor, touchscreen, projector, or other display device known in the art, which may enable a user to view data in raw form, in graphs and/or timelines, or in another form, and/or interact with various data stored in or downloaded from non-transitory memory 106.

In one example, wearable fall risk monitoring device 101 may be worn on a wrist, hip, or ankle of the wearer, and display device 134 may be a wearable display device, such as a screen positioned on the wearable fall risk monitoring device 101, such that the wearer may view alerts or notifications displayed on the screen in regard to a risk of falling. In another example, display device 134 may comprise a computer monitor, and may display notifications indicating outputted analyses and/or raw data (e.g., graphs, timelines, etc.). For example, the wearer may connect the wearable fall risk monitoring device 101 to a computer of the wearer, and the outputted analyses and/or raw data may be displayed to the wearer on the computer monitor. As another example, the wearable fall risk monitoring device 101 may transmit the outputted analyses and/or raw data to a display device 134 of a computing device of a healthcare professional, a physical trainer (PT), a workplace supervisor, or another type of professional at a remote device 135, who may review the outputted analyses and/or raw data and provide feedback to the wearer, initiate a professional consultation with the wearer, or initiate another method of intervention.

The fall risk assessment system herein disclosed thus implements specialized sensor fusion and machine learning techniques that enable real-time preventative fall risk assessment. The system integrates multiple sensor types and processing techniques to more proactively identify potential fall hazards across generalizable populations and scenarios. Further, the system may integrate data from multiple devices to build a fall risk exposure event map that may further aid in preventing falls before they occur and/or provide geolocations of events for hazard modification purposes.

The fall risk assessment system aims to prevent falls through a sensor fusion architecture that combines IMU motion data with, for example, electrodermal activity biosensor data in a way that reduces false positives compared to systems that rely on motion sensing alone. The synchronized multi-sensor approach enables more accurate fall risk assessment by correlating physiological arousal with abnormal movement patterns. Additionally, the unsupervised learning approach employed for the machine learning model may allow for processing complex time-series data without requiring extensive labeled training data. This may provide increased accuracy across a wider range of walking or other movement conditions compared to supervised learning approaches. Further, the system enables real-time signal processing techniques for analysis of biosensor data, including convex optimization-based decomposition, that enables the system to quickly identify meaningful physiological responses while filtering out noise and interference.

Integration of GPS data with the fall risk assessments to generate detailed hazard maps provides a concrete method of identifying and avoiding fall hazards in the physical environment. For example, by acquiring fall risk score data from a plurality of devices, the system can generate an interactive map that is accessible to individual users. The map may indicate locations of potential hazards that may aid users in avoiding those hazards or locate areas to target interventions that reduce the fall hazards, as will be further described herein.

It should be understood that fall risk assessment system 100 shown in FIG. 1 is for illustration, not for limitation. Other appropriate monitoring systems may include more, fewer, or different components.

Referring to FIG. 2, an exemplary fall risk assessment model training system 200 is shown, which may be used to train a fall risk assessment model 220. The fall risk assessment model 220 may be used by a fall risk assessment system, such as the fall risk assessment system 100 of FIG. 1. The fall risk assessment model 220 may be stored within a ML model module 242 of the fall risk assessment system, which may be a non-limiting example of the ML model system 108 of fall risk assessment system 102 of FIG. 1. The fall risk assessment model 220 may be trained to identify abnormal movements indicative of potential exposure to fall hazards, based on time-series sensor data 202 (e.g., sensor data 114 of FIG. 1) collected by a fall risk monitoring device 250, which may be the same as or similar to the fall risk monitoring device 101 of FIG. 1. The time-series sensor data may also be referred to herein as sensor data.

In various embodiments, the time-series sensor data 202 may include a continuous stream of changes in measurements acquired from an IMU 251 and/or one or more sensors 252 (such as IMU 136 and one or more additional sensors 140 of FIG. 1) of the fall risk monitoring device 250 over time, in real time, as a wearer of the fall risk monitoring device 250 moves throughout an environment on foot. The time-series sensor data 202 may include gait, stride, and/or other body movement data collected as the wearer walks or runs. The time-series sensor data 202 may be collected as the wearer moves across various surfaces of different types, materials, and/or degrees of incline. For example, the various surfaces may include flat surfaces, sloping surfaces, rough surfaces, smooth surfaces, slippery surfaces, stairs, ladders, etc., that may be encountered by a person during walks or other activities. For example, the time-series sensor data 202 may be collected from a variety of patients of a healthcare system or from a variety of employees of a workplace.

The fall risk assessment model training system 200 may include a time series window generator 203, which may extract portions of time-series sensor data 202 corresponding to discrete time series windows 206. For example, time series window 206 may comprise 2 seconds of time-series sensor data, 5 seconds of time-series sensor data 202, or 10 seconds of time-series sensor data 202, or several minutes of time-series sensor data 202, or a different amount of time. During training of the fall risk assessment model 230, the time series window generator 203 may continuously extract time series windows 206 from one or more subjects included in time-series sensor data 202. For example, time-series sensor data 202 may include a first set of sensor data from a first subject, from which a first set of time series windows 206 is extracted; a second set of sensor data from a second subject, from which a first set of time series windows 206 is extracted; a third set of sensor data from a first subject, from which a first set of time series windows 206 is extracted; and so on. Durations of the first set of sensor data, the second set of sensor data, and the third set of sensor data may be equal, or may not be equal. Additionally, time series windows 206 may be overlapping time series windows. The first set of time series windows 206, the second set of time series windows 206, and the third set of time series windows 206 may be used to train the fall risk assessment model 220. In this way, fall risk assessment model 220 may be trained on time-series sensor data collected from the subject wearing the wearable monitoring device.

During extraction of the time series windows 206 from the time-series sensor data 202, portions of time-series sensor data 202 may be stored in a data array in a buffer in a memory of the fall risk assessment system (e.g., sensor data 114 of fall risk assessment system 100), and each time series windows 206 may be selected from the data array based on an initial time reference, as described below in reference to FIGS. 3, 5, and 6.

Additionally, one or more subject parameters 207 may be additional inputs into the fall risk assessment model during training. The parameters may include, for example, an age of a subject; a weight of the subject; a disability of the subject; a rating or numerical score indicating a degree of mobility of the subject; and so on. It should be appreciated that the examples provided herein are for illustrative purposes, and other types of parameters may be included without departing from the scope of this disclosure.

The fall risk assessment model 220 may be trained using the time series windows 206 and potentially the subject parameters 207 via unsupervised learning. During training, the time series windows 206 may be mapped onto graph-structured data, which may capture nonlinear and complex relationships between data points in the time-series sensor data 202. In this way, abnormal body movements that may be indicative of an increased risk of a fall may be distinguished from subtle variations in repetitive regular movements. An exemplary method for training the fall risk assessment model 220 is described below in reference to FIG. 3.

In some examples, data processing may be executed via a microcomputer within the fall risk monitoring device. In other examples, data processing may be executed remotely via a device (e.g., a smart phone, smart watch, etc.) communicatively coupled to the microcomputer within the fall risk monitoring device.

Further, data augmentation techniques may be employed to further enrich the dataset by taking advantage of invariants and symmetries (e.g., spatial and/or temporal) in the data. As an example, gathering enough real data of falls of subjects may be difficult, and as such simulated data may be included in the training data to increase robustness thereof. In addition to the graph-based analysis, methods such as principal component analysis (PCA)-based anomaly scoring, local outlier factor, and/or one-class support vector machine may be employed for detection of anomalous patterns in fall risk monitoring device sensor data (e.g., motion measurements, orientation measurements, electrodermal, heart-rate measurements, and the like).

Referring now to FIG. 3, a flowchart illustrating a method 300 for training a fall risk assessment model, such as the fall risk assessment model 220 of the fall risk assessment model training system 200 of FIG. 2, according to an exemplary embodiment. Method 300 may be executed by a processor of a fall risk assessment system, such as processor 104 of the microcomputer 102 of the fall risk assessment system 100 of FIG. 1. In one example, some operations of method 300 may be stored in non-transitory memory of the fall risk assessment system, for example in a training module (such as the training system 110 of memory 106 of FIG. 1) and executed by the processor of the system.

Method 300 begins at 302, where method 300 includes receiving time-series sensor data of a human subject. The time-series sensor data may be body movement data collected by sensors of a fall risk monitoring device, such as the fall risk monitoring device 101 of FIG. 1, and may include an IMU with a plurality of motion sensors, including accelerometers, gyroscopes, and/or magnetometers. In some examples, the fall risk monitoring device may further comprise one or more other sensors, such as optical sensors, temperature sensors, and/or other types of sensors. Acquiring body movement data may comprise employing the IMU to acquire motion data and orientation data. The body movement data may be acquired while the subjects are walking, running, exercising, or performing other tasks involving physical movement supported by feet of the subjects.

The body movement data acquired may include subtle and large movements and orientation changes as well as intentional, unintentional, spontaneous, and deliberate movements and orientation changes of a body part of a subject on which the fall risk monitoring device is worn. The fall risk monitoring device may include known limits of an extension, flexion, and lateral movement of the body part as based on anatomical limits. Acquired data that is outside of the anatomical limits may indicate to the system that drift or other abnormal acquisition has occurred. If detected, a notification may be presented on a display device, for example a remotely connected display device, indicating that re-calibration of the IMU is indicated. In this way, more accurate motion data may be provided by the systems herein described.

At 304, method 300 includes generating (e.g., extracting data values of the time-series sensor data into) a plurality of overlapping time series windows (e.g., time series windows 206) of a predefined time. For example, the predefined time may be 2 seconds. The time series windows may be created as described above in reference to FIG. 2.

For example, the time-series sensor data may include a relative position of the fall risk monitoring device in three dimensions at a time t1. The relative position may be represented as a position vector including multiple individual data values indicating a change in a position of the fall risk monitoring device in three dimensions from a previous position at a time t0. A first time-series window of, for example, 2 seconds may be created from the time-series sensor data. The first time-series window may include 60 position vectors per second, each position vector including three data values indicating a change in a position of the fall risk monitoring device. The first time-series window may start with a first position vector and end with a 120^{th} position vector of the time-series sensor data. The data values included in each position vector may be collected from a corresponding subject at the predetermined time interval.

A second time series window of, for example, 2 seconds may be created from the time-series sensor data at a time t2 including 120 position vectors, starting with a second position vector and ending with the 101^{st} position vector of the time-series sensor data, such that the second time-series window is offset from the first time-series window by one position vector, and overlaps the first time-series window when plotted on a timeline. A third time-series window of 2 seconds may be created from the time-series sensor data at a time t3 including 120 position vectors, starting with a third position vector and ending with an 103^{th} position vector of the time-series sensor data, such that the third time-series window is offset from the second time-series window by one position vector and from the first time-series window by two position vectors, and overlaps the first time series window and the second time series window when plotted on the timeline, and so on.

It should be appreciated that for simplicity, a time series window of only position vectors is described above. In various embodiments, a time series window may include additional data, such as an orientation vector of individual data values indicating an orientation of the fall risk monitoring device in three dimensions at a time tₙ, including multiple directions of accelerometry, goniometry, and magnetometry, plus features extracted from physiological sensors such as EDA, PPG (optical), and ECG sensors as well as body and environmental temperature and light levels; a data value indicating a level of light in the environment at the time tₙ; and/or other data that may be a factor influencing a risk of the wearer falling. This may allow for multiple modes and dimensions of movement sensing, enabling a more accurate determination of fall risk versus other movement types, and EDA and PPG/ECG measurements allow measurement of the body's physiological responses to fall hazards. Further, if a measured temperature of the wearer is above 98.6 degrees, it may be inferred that the wearer has a fever, and may experience dizziness; if a measured temperature of the environment is above a threshold temperature, it may be inferred that the wearer may become exhausted more rapidly and may lose balance; if a measured level of light is below a threshold, it may be inferred that the wearer may have difficulties seeing changes in a surface the wearer is walking on or obstacles in a path of the wearer; and so on.

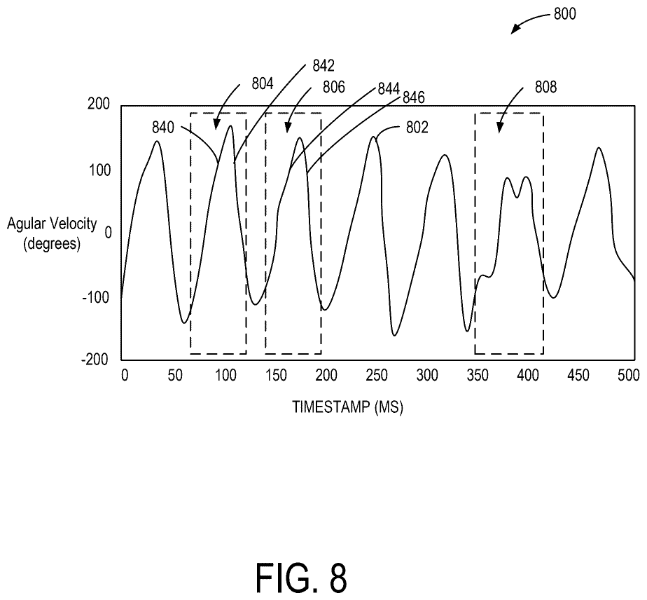

Turning briefly to FIG. 8, a time series diagram 800 shows an exemplary time series body movement data of a subject acquired while the subject is wearing a fall risk monitoring device on a wrist while walking, plotted on a line 802. A change in relative position of the fall risk monitoring device in one dimension is represented as angular velocity on the y-axis, and time in milliseconds on the x-axis. The time-series sensor data may be collected by an IMU of the wearable device continuously from the subject over a period of time.

A depicted portion of the time-series sensor data includes a first step 804 with a first foot, and a second step 806 with the same first foot (e.g., for a total of four steps accounting for both feet). That is, during a first portion 840 of the time-series sensor data, an arm of the subject may be swinging in a first direction as the subject takes first step 804. During a second portion 842 of the time-series sensor data, the arm of the subject may swing in an opposite second direction as the subject takes a step with the opposite foot. Similarly, during a third portion 844 of the time-series sensor data, the arm of the subject may swing in first direction as the subject takes second step 806 with the first foot. During a fourth portion 846 of the time-series sensor data, the arm of the subject may again swing in the opposite second direction as the subject takes a step with the opposite foot. The arm swing in the first and second directions may follow a generally cyclical pattern, where the arm swings to roughly the same angular velocity with each swing. This cyclical pattern may be predictable for a normal or typical/baseline walking pattern.

The time-series sensor data further includes a region 808 that corresponds to an abnormal step. For example, the typical swing in the first direction followed by a swing in the second direction is not seen. The movement of the arm at this point may be detected by a fall risk assessment model as an abnormal movement. Taken alone, the abnormal movement could indicate a disruption of a gait or stride of the wearer, such as when exposed to a fall hazard. However, the abnormal movement could also be a result of the wearer moving their arm in a non-walking motion, such as to point at something in the environment, scratch their nose, tug on a dog leash that they are holding, or any other number of actions. When such an abnormal movement coincides with abnormal physiological arousal, thereby indicating exposure to a fall hazard, the wearer may be notified by the fall risk monitoring device of a potential increase in a risk of falling.

Individual position values may be extracted at regular, predefined intervals from the time-series sensor data, to generate a data array that may be continuously added to over time. The extracted data values may then be further extracted into a plurality of overlapping time series windows, which may be inputted into the fall risk assessment model to detect the abnormal movement.

At the passage of each predefined interval, a new time series window may be inputted into a fall risk assessment model. The fall risk assessment model may learn to perform sliding window inferences on the data included in the time series windows over various inference intervals, where a single time series window of sensor data is inputted into the fall risk assessment model at each inference interval. It should be appreciated that data array 508 is a one-dimensional array for purposes of simplicity, and that the time series windows may include multi-dimensional data such as position and orientation vectors, and other sensor data.

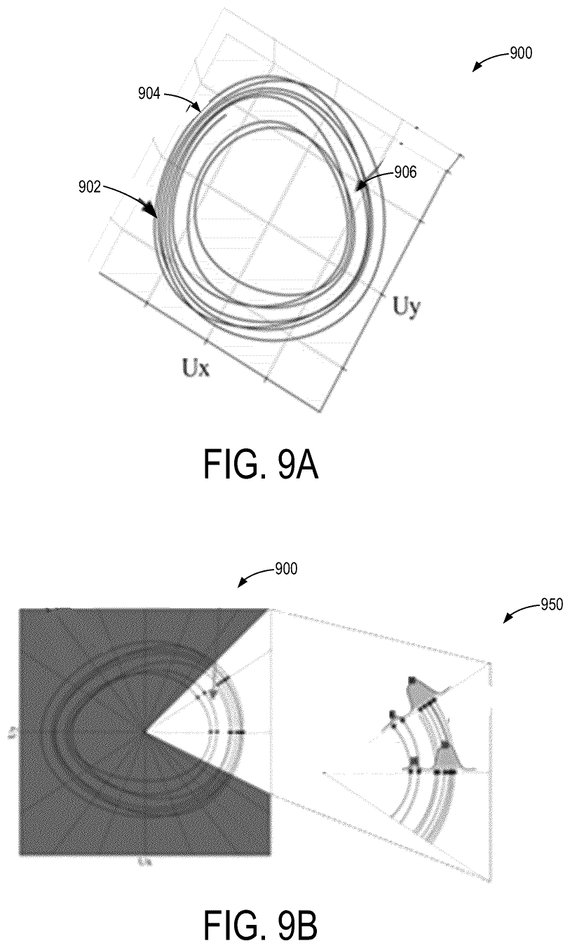

To learn from each individual's data rather than comparing their data to historical datasets, the data provided by the IMU of the wearable device is transformed into nodes and edges. For example, turning briefly to FIGS. 9A and 9B, a transformation 900 of angular velocities is shown. The transformation 900 may be generated from angular velocity data of the wearable device, such as the data presented in time series diagram 800 of FIG. 8. Each data point of the data is transformed into a node, wherein the relationships between nodes are edges. Nodes can have multiple relationships to other nodes, allowing for complex, non-linear relationships. Quantification of the strength of these relationships allows for identification of anomalies in the data pattern. For example, strength of relationships for typical/baseline movement data may be relatively stable or constant, while strength of relationships for abnormal movement data may be notably different from the baseline strength for typical movement data.

As an example, in the transformation 900, first and second sections 902 and 904 correspond to data of typical or baseline walking movements, such as first and second steps 804 and 806 described with respect to FIG. 8. In contrast, a third section 906 may correspond to data of atypical or abnormal body movement, such as the region 808 of data of the time series diagram 800. FIG. 9B shows the third section 906 in greater detail in an enlarged section 950. As an example, subsequences that cross a section of the 2D space of the transformation 900 are evaluated, creating a node at the local distribution maxima. By connecting nodes by edges, a node weight by the number of edges that connect to it may be determined. Similar subsequences generate larger node degrees and edge weights compared to abnormal subsequences. This may be quantified with a normality score representing the sum of edge weights and node degrees between adjacent nodes over the length of data points, as represented in equation (1):

$$\\ \begin{matrix}{{\sum }_{j = 1}}^{i + l - 1} \frac{w ( {N}_{j} , {N}_{j + 1} ) deg ( {N}_{j} - 1 )}{l} & ( 1 )\end{matrix}$$

where, w(Nⱼ, Nⱼ₊₁) is edge weight, deg(Nⱼ) is node degree, i is the ith subsequence, and l is the query length.

Returning to method 300, at 306, method 300 includes training the fall risk assessment model on the overlapping time series windows. The fall risk assessment model may be trained using unsupervised learning. Using the unsupervised graph-based approach may allow for detection of fall hazards without demanding large training datasets that represent every type of person, task, and environment. This approach allows for learning from each individual's data rather than comparing their data to historical datasets. As herein described, data may be evaluated along 2 second windows, advancing one data point at a time. Local convolution of 40-data-point windows, followed by principal components analysis may be used to project the data into a 2-dimensional space. Then the algorithm may evaluate subsequences that cross a section of the 2D space, creating a node at the local distribution maxima. Nodes can be connected by edges, getting a node weight by the number of edges that connect to it. Similar subsequences generate larger node degrees and edge weights compared to abnormal subsequences. A normality score may represent the sum of edge weights and node degrees between adjacent nodes over the length of data points. A similar process may be applied to the frequency spectrum characteristics of the signal, for example, using the average power spectral density, which decreases when hazard exposed.

Once the model is trained, during analysis of time-series data, the body movement sensor data included in the overlapping time series windows may be mapped onto graph-structured data, which may capture nonlinear and complex relationships between data points in the body movement sensor data.

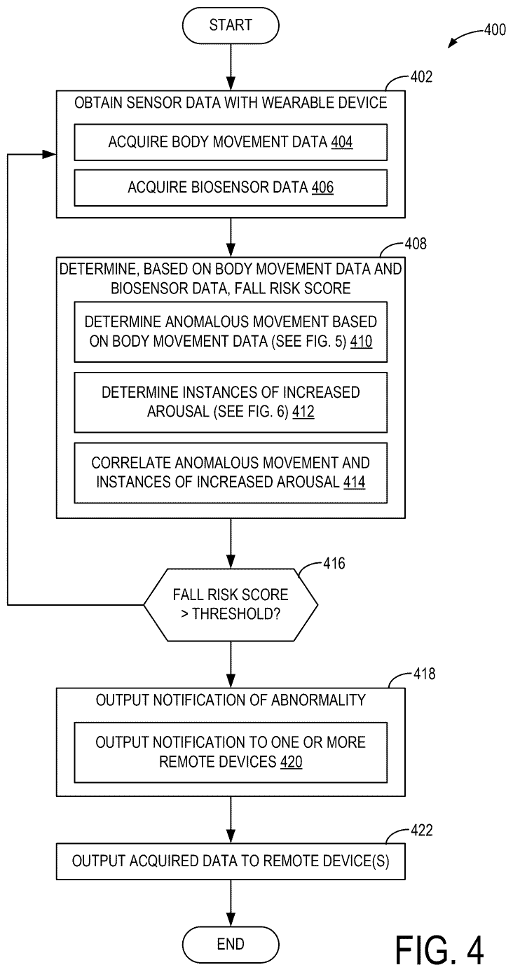

Referring now to FIG. 4, a flowchart illustrating a method 400 for identifying fall risk exposure events using acquired sensor data, including body movement data acquired by an IMU of a wearable fall risk monitoring device and physiological arousal data acquired by one or more biosensors such as EDA sensors, is shown. Method 400 may be executed by a processor of a fall risk assessment system, such as processor 104 of the fall risk assessment system 100 of FIG. 1. At least some operations of the method 400 may be stored in a non-transitory memory of the fall risk assessment system (e.g., in non-transitory memory 106) and executed by the processor.

At 402, method 400 includes obtaining sensor data with a wearable device, such as a wearable fall risk monitoring device. Obtaining the sensor data may include acquiring body movement data of a wearer of the wearable fall risk monitoring device that includes a plurality of sensors, as noted at 404. For example, the body movement data may be acquired by an IMU integrated into the wearable device. In some embodiments, body movement data may be acquired and processed in real-time, without being stored in memory of a microcomputer of the fall risk monitoring device. In other embodiments, the data may be additionally, selectively, and/or temporarily stored in the memory for later processing and/or transmission. In some examples, the body movement data may be acquired at a sampling frequency of 60 Hz, which may allow for adequate capture of details of motion and orientation while effectively managing data volume, though other sampling frequencies are possible.

Obtaining sensor data may further include acquiring biosensor data of the wearer of the fall risk monitoring device, as noted at 406. The biosensor data may include data acquired by one or more biosensors of the plurality of sensors integrated into the wearable device. In some examples, the one or more biosensors may include EDA sensors that are configured to detect changes in electrical activity of the wearer's skin resulting from changes in sweat gland activity. Changes in sweat gland activity, as is herein described, may be an indicator of physiological arousal, wherein peaks in sweat gland activity correspond to increased arousal. The body movement data and the biosensor data may be acquired simultaneously as time series data.

It should be appreciated that the biosensor data and the body movement data may be acquired by the wearable device concurrently. Both the biosensor data and the body movement data may be timestamped to allow for time aligning between the two. Further, it should be appreciated that while a single fall risk monitoring device is described herein, more than one fall risk monitoring device of the present disclosure may be worn by the wearer at the same time. For example, the wearer may wear one fall risk monitoring device on one wrist and a second fall risk monitoring device on the other wrist, or the wearer may wear one fall risk monitoring device on one wrist and a second fall risk monitoring device on one ankle, or on a different part of the wearer's body. As such, sensor data may be acquired from multiple sources and may be inputted for processing together to provide for more robust analysis.

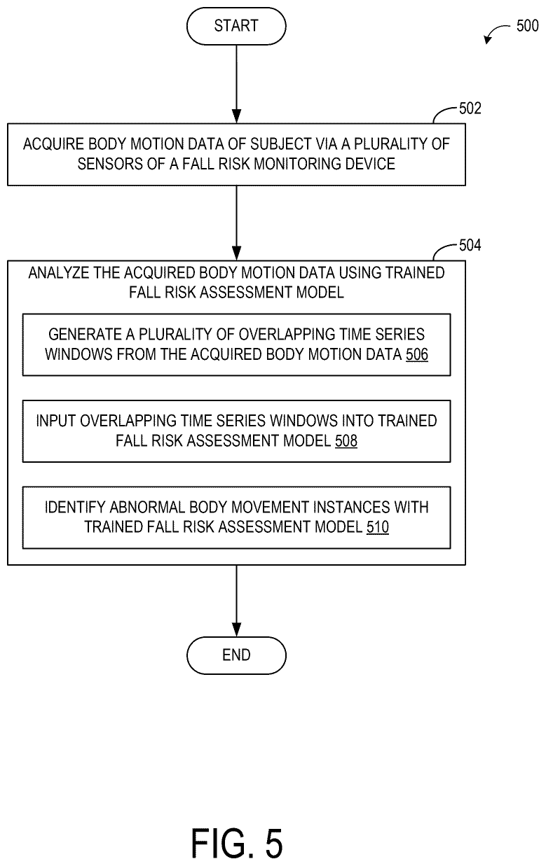

At 408, method 400 includes determining, based on the body movement data and the biosensor data, a fall risk score. Determining the fall risk score may include determining anomalous movement based on the body movement data, as noted at 410, determining instances of increased arousal, as noted at 412, and correlating the anomalous movement and the instances of increased arousal, as noted at 414. Determining anomalous movement may include deploying a trained fall risk assessment model, as will be further described with respect to FIG. 5. The fall risk assessment model may be the trained fall risk assessment model 230 described with respect to FIG. 2. In various embodiments, the fall risk assessment model may be trained as described above in reference to the method 300 of FIG. 3. Determining instances of increased arousal may include signal smoothing, which includes decomposing the smoothed data signals to isolated high-frequency signals, and then identifying anomalous high-frequency signals, as will be further described with respect to FIG. 6.

Correlating the instances of anomalous movement and the instances of increased arousal may include time aligning the body movement data and the biosensor data. This may include time aligning respective abnormal instances of body movement and biosensor data to determine whether the instances occur at relatively the same time, such as within a predefined threshold such as within 100 ms, or some other threshold. The buffer threshold may account for a delay in physiological arousal following an exposure. For example, wearer may begin to lose their balance when walking on uneven ground. Abnormal body movement may be seen first, in a time series, and then after a brief delay, the physiological arousal may increase in response to the abnormal body movement.

The fall risk score may be determined by the modeled score from the movement data, from the biosensor data, or a combined score from both movement and biosensor data and may indicate a likelihood that the wearer was exposed to a fall hazard. Although each sensor modality can generate a fall risk score if recorded alone, the combination improves the accuracy to identify a true fall hazard and reduces the potential for false positive or false negative events. For example, baseline body movement data and baseline biosensor data, evaluated in combination at a first time, may indicate a low fall risk score. Abnormal body movement data evaluated in combination with baseline biosensor data at a second time may indicate a medium fall risk score. Similarly, abnormal biosensor data evaluated in combination with baseline body movement data at a third time may indicate a medium fall risk score. Thus, the fall risk score from a sensor modality alone may not necessarily represent true fall risk. Thus, evaluating fall risk based on a combined evaluation of abnormal movement data and abnormal biosensor data may mitigate erroneously identifying a fall risk from abnormal body movement or abnormal physiological responses alone. Abnormal body movement data evaluated in combination with abnormal biosensor data at a fourth time may indicate a high fall risk score. For example, the fall risk score may be determined on a scale of 0 to 1, whereby a fall risk score of 0 indicates low risk for falls and a fall risk score of 1 indicates high risk for falls. Various thresholds on the scale between 0 and 1 may be defined as low, medium, high risk, in some examples. For example, a score between 0 and 0.3 may be considered low risk, a score between 0.3 and 0.6 is considered medium risk, and a score between 0.7 and 1 is considered high risk, categorically. Predefined thresholds for what is considered a fall risk exposure event may also be defined. For example, any fall risk score above 0.7 may be considered to indicate a fall risk exposure event. For example, the fall risk score may be computed by multiplying the abnormality index (calculated by IMU data through the graph-based method) and the anomaly index (calculated by EDA data through the convex optimization method). At 416, method 400 includes determining whether the fall risk score is greater than a predefined threshold. Fall risk scores greater than predefined threshold may indicate that the data to which the fall risk score corresponds indicate a fall risk exposure event. If the fall risk score is greater than the predefined threshold, method 400 proceeds to 418. If the fall risk score is less than the predefined threshold, method 400 returns to 402 to continue acquiring sensor data with the wearable fall risk system.

At 418, method 400 includes outputting a notification of the abnormality. For example, a notification indicating that the wearer is at increased risk of falling may be outputted to the wearer of the wearable device. The type of notification and where the notification is outputted may vary. The notification may be outputted to the wearer of the wearable device via a display device (e.g., display device 134 of FIG. 1) integrated into the wearable device itself, or to a haptic feedback system (e.g., haptic feedback system 144), which may notify the wearer via haptic feedback. For example, the haptic feedback may include a vibration, or a temperature change, or a different type of haptic feedback. In some examples, the notification may be outputted to a speaker of the wearable device or to a remote device communicatively coupled to the wearable device, where the wearer may be notified by audio signals such as tones or speech. Additionally, the notification may be graded based on an estimated level of fall hazard exposure or a probability that the wearer might fall, in some examples. For example, a first fall risk score corresponding to the first abnormal body movement may be detected, and that fall risk score may be combined with a fall risk score derived from a first anomalous physiological arousal, from which a first display/haptic feedback/tone notification may be generated. A second, higher fall risk score corresponding to a more pronounced abnormal body movement and/or higher anomalous physiological arousal may be detected thereafter within a predefined amount of time, and a second display/haptic feedback/tone notification may be generated that is different from the first notification.

The notification may be additionally or alternatively outputted to a connected remote device for display, as noted at 420, such as the wearer's smart phone, and/or to one or more other remote devices, such as a healthcare provider of the wearer, a family member of the wearer, a workplace supervisor, etc. For example, the fall risk monitoring device may have one or more settings turned on or off that indicate parties to be contacted in the event of a detection of an increased risk of the wearer falling, or a pattern of behavior of increased risks over time.

At 422, method 400 includes optionally outputting the acquired sensor data, including both the body movement data and the biosensor data, to a remote device. In some examples, the wearable fall risk monitoring device through which the data is acquired may have an internal memory of a microcomputer (e.g., memory 106 of the microcomputer 102 of FIG. 1). The internal memory of the wearable device may store some or all of the acquired data, which may be processed and analyzed by the microcomputer at a later time. Further, it should be understood that in some examples the data may be offloaded to the remote device for analysis rather than having the analysis of the data be performed by the wearable device itself. In such examples, the stored acquired data may be outputted from the wearable device to a remote device for long term storage and/or analysis, for example, for inclusion in a research study. Outputting the acquired data may occur at regular intervals, upon manual request, or when data storage of the internal memory of the wearable device is full, or some combination thereof. The acquired data may be outputted both in raw form and in analyzed form, in some examples. The acquired data may be accessible via the remote device to which it was outputted for additional analysis. Analysis of data over time may allow for determination of response to therapy, including physical/occupational therapy as well as medication therapy, changes in mental status or cognitive function, environmental interventions in the built environment or workplace, and more. In this way, the processing efficiency of the fall risk monitoring device may be maintained and the acquired data may be accessible on a long term basis.

In yet further examples, as will be further described with respect to FIG. 8, the identified abnormal instances may be geolocated to generate a map of fall risk exposure events. User inputs, such as uploaded photos, indications of a fall risk exposure, and the like, may be accessible from within the generated exposure map.

It should be appreciated that method 400 may be executed in a continuous, iterative manner, whereby data is continuously acquired and analyzed in real-time or near real-time. Notifications may be outputted and displayed for relevant portions of the acquired body movement data in real-time as well. Further, data may continue to be acquired by the sensors while a notification is outputted and/or displayed.

Turning now to FIG. 5, a flowchart is shown illustrating a method 500 for identifying abnormal body movement from acquired body movement data using a trained fall risk assessment model, such as the trained fall risk assessment model 230 of FIG. 2. Method 500 may be executed by a processor of a fall risk assessment system, such as the fall risk assessment system 100 of FIG. 1. Some operations of method 500 may be stored in a non-transitory memory of the fall risk assessment system (e.g., in inference system 112 of the microcomputer 102 of FIG. 1) and executed by the processor. In various embodiments, the fall risk assessment model may be trained as described above in reference to the method 300 of FIG. 3.

At 502, method 500 includes acquiring body movement data of a wearer of a fall risk monitoring device including a plurality of sensors, as described above. In some embodiments, the body movement data may be acquired and processed in real-time, without being stored in memory of a microcomputer of the fall risk monitoring device. In other embodiments, the data may be additionally and/or selectively stored in the memory (e.g., sensor data 114 or storage device 133) for later processing and/or transmission. In some examples, the data may be acquired at a sampling frequency of 60 Hz, which may allow for adequate capture of details of motion and orientation while effectively managing data volume, although other sampling frequencies are possible.

In some examples, the acquired body movement data may be offloaded or downloaded in real time to a remote device including a trained fall risk assessment model, such as a workstation, desktop computer, a smart watch, a smart phone, etc., for processing of the body movement data. Offloading the acquired data to the remote device may reduce computational demands of the fall risk monitoring device and therefore may increase longevity of a battery of the fall risk monitoring device. Alternatively, the acquired data may be analyzed by the fall risk assessment model at the fall risk monitoring device. Further, in some examples, a portion of analysis may be employed at the fall risk monitoring device, such as timing of events, while other portions of the analysis may be performed by the remote device.

At 504, method 500 includes analyzing the acquired body movement data using the trained fall risk assessment model. At 506, analyzing the acquired data using the trained fall risk assessment model includes generating a plurality of overlapping time series windows from the acquired body movement data, as described above in reference to FIGS. 3 and 8. At 508, analyzing the acquired body movement data using the trained fall risk assessment model includes inputting each time series window of the overlapping time series windows into the trained fall risk assessment model at a corresponding time interval. The trained fall risk assessment model may output an estimated probability of the wearer falling or otherwise a fall risk score indicating a level of exposure to a fall, based on the acquired body movement data included in the overlapping time series windows. In other words, the trained fall risk assessment model may perform sliding window inferences on the acquired body movement data at the regular inference intervals at which the time series windows are inputted into the trained fall risk assessment model.

Each time-series window may contain high-dimensional data. This data is reduced to three dimensions using, for example, Principal Component Analysis (PCA) and then projected onto a 2D plane by projecting along the z-axis (e.g., the temporal axis). Thus, the high-dimensional data may be represented on a 2D plane. On the 2D plane, the data is transformed into a graph structure and the abnormality score (e.g., the inverse of the normality score) is calculated based on equation (1) described above within the graph structure. For each sliding window, the same process is applied to the subsequent time series window, repeating the process to calculate the abnormality score for the entire sequence.

To analyze the body movement data, which may be acquired as angular velocities with respect to time, the model may transform the angular velocities into groups of nodes and edges. Quantification of the relationships between nodes and edges may allow for identification of abnormal instances in the data vs chunks of typical or baseline data, as described above with respect to FIGS. 9A and 9B.

At 510, analyzing the acquired body movement data using the trained fall risk assessment model may include identifying abnormal body movement instances with the trained fall risk assessment model based on the time series windows. Instances identified as abnormal body movements may indicate that the wearer has been exposed to a fall hazard or a higher probability of falling. The abnormal body movement index from IMU data, derived through a graph-based method, can independently serve as an indicator of the fall hazard exposure or probability of falling. However, multiplying this index by the anomalous arousal index calculated from, for example, EDA data allows for a more reliable measurement of the probability of falling. Therefore, this combined value is used as the final output of the fall risk score to represent the likelihood of falling.

As is described with respect to method 400, the instances of abnormal body movement and/or the moments of increased probability of falling may be considered along with analyzed biosensor data to determine a fall risk score. Thus, the method 500 may be incorporated as part of a larger method for fall risk assessment, such as at 408 and 410 of method 400. It should be appreciated that method 500 may be executed in a continuous, iterative manner, whereby data is continuously acquired and analyzed in real-time or near real-time. For example, the method 500 may be executed for sliding windows of data, as previously described.

Turning now to FIG. 6, a flowchart is shown illustrating a method 600 for analyzing biosensor data acquired with a wearable fall risk monitoring device of a fall risk assessment system, such as the fall risk assessment system 100 of FIG. 1. Method 600 may be executed by a processor of the fall risk assessment system, such as the processor 104 of the microcomputer 102 of FIG. 1. Some operations of method 600 may be stored in a non-transitory memory of the fall risk assessment system (e.g., in inference system 112 of the microcomputer 102 of FIG. 1) and executed by the processor. It should be understood that the method 600 is merely exemplary and other methods for analyzing biosensor data to determine physiological arousal and identify instances of anomalous arousal may be employed. Further, it should be appreciated that the method 600 may be integrated as part of a method for fall risk assessment, such as at 408 and 412 of method 400 described above.

At 602, method 600 includes acquiring biosensor data with one or more biosensors of the wearable fall risk monitoring device. In some examples, the one or more biosensors may include EDAs that are configured to detect changes in electrical activity of the wearer's skin resulting from changes in sweat gland activity. Changes in sweat gland activity, as is herein described, may be an indicator of physiological arousal, wherein peaks in sweat gland activity correspond to increased arousal. In examples in which the one or more biosensors include EDA sensors, the biosensor data may be acquired in micro-Siemens (e.g., resistance to the current passed through the skin in units of conductance).

At 604, method 600 includes decomposing the biosensor data to isolate high-frequency signals. Decomposing the biosensor data may be a signal smoothing technique that aims to filter out irregular fluctuations caused by electronic interference and environmental noise. The high-frequency signals may represent acute, short-term arousal responses to stimuli, such as those that occur in response to a person losing their balance.

In some examples, decomposing the biosensor data may include applying a convex optimization-based decomposition, as noted at 606. In general, convex optimization-based decomposition may include decomposing a signal into a smooth, low-frequency component and a high-frequency component. The smooth component may represent trend-like data without rapid fluctuations and the high-frequency component may represent rapid fluctuation, such as rapid onset peaks in signal. The decomposition uses convex optimization to ensure efficient and globally optimal outputs, such as minimizing variation between adjacent points for the smooth component. The convex optimization-based model applied in this invention is represented as follows in equation (2):

$$\\ \begin{matrix}{min}_{q , l , d} \frac{1}{2} {‖ Mq + Bl + Cd - y ‖}_{2}^{2} + α {‖ Aq ‖}_{1} + \frac{γ}{2} {‖ l ‖}_{2}^{2} & ( 1 )\end{matrix}$$

$$\\ Subject to Aq \geq 0$$

Where M, B, C, A are transformation metrics, q is the high frequency component, l is the low frequency component, d is the residual term, and y is the observed signal.

Decomposing the signal to isolate high-frequency signals may further include summing the amplitudes of the high-frequency component over a specified time window, as noted at 608. Summing over the time window may indicate the level of physiological response. In some examples, the time window of 10 s is used for summing the amplitudes of the high-frequency components. Other time window durations may be selected. Summing the amplitude over the time window may thus indicate a level of response for the given time window, wherein if the level of response is above a predefined threshold, anomalous physiological arousal may be detected for that given time window.

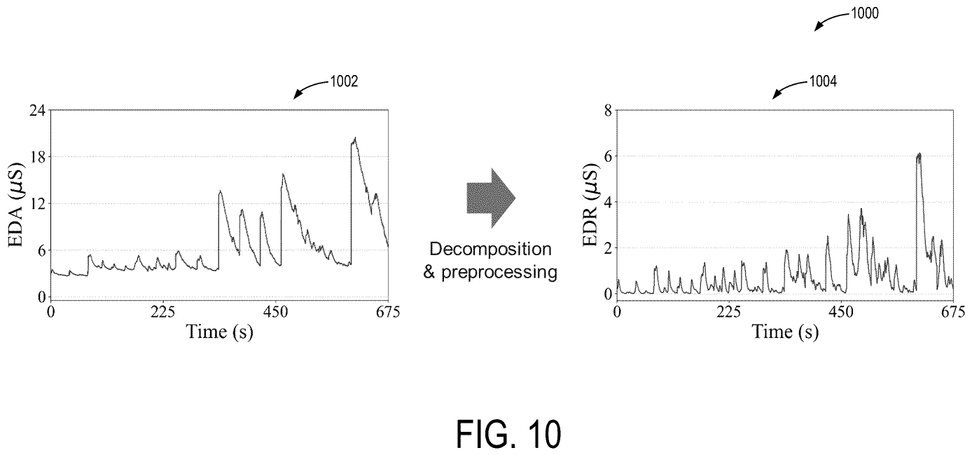

Turning briefly to FIG. 10, a decomposition 1000 is shown. First graph 1002 shows raw data acquired by a biosensor, such as an EDA, of a wearable fall risk monitoring device. Second graph 1004 shows decomposed and preprocessed data corresponding to the raw data. As shown, a baseline electrodermal activity in the raw data may be around 3 micro-Siemens and include slow, low-frequency changes in amplitude. In the decomposed data, the baseline offset is removed to be around or just above 0 micro-Siemens, and the low-frequency components of the data are filtered out. This may allow for more accurate identification of transient electrodermal responses to stimuli such as a loss of balance, thereby allowing for more accurate detection of relevant abnormal physiological arousal.

Returning to FIG. 6, at 610, method 600 includes determining anomalous high-frequency signals. Based on the summing, anomalous high-frequency signals may be identified. For example, a threshold summed value for the predefined time window may be known. Summed values that exceed the threshold may be considered anomalous, thereby indicating that the high-frequency components of the decomposed signals are indicative of increased physiological arousal.

Similar to the analysis of body movement data, as is described with respect to method 400, the instances of anomalous biosensor data may be considered along with analyzed body movement data to determine a fall risk score. It should be appreciated that method 600 may be executed in a continuous, iterative manner, whereby data is continuously acquired and analyzed in real-time or near real-time (e.g., with sliding time windows).

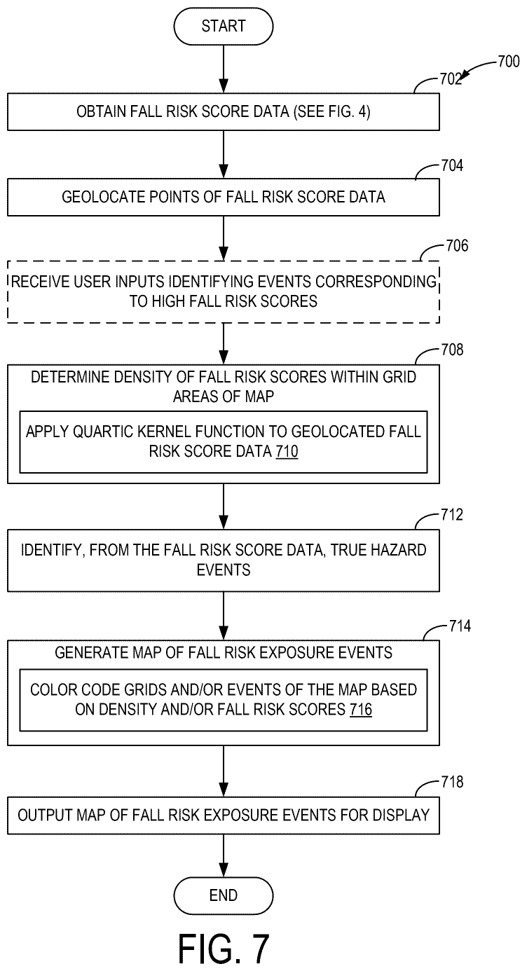

Turning now to FIG. 7, a flowchart illustrating a method 700 for geolocation of fall risk exposure events is shown. The method 700 may be executed by a processor of the fall risk assessment system, such as the processor 104 of the microcomputer 102 of FIG. 1. Some operations of method 700 may be stored in a non-transitory memory of the fall risk assessment system (e.g., in the fall risk geolocation system 118 of the microcomputer 102 of FIG. 1) and executed by the processor.

At 702, method 700 includes obtaining fall risk score data. As described with respect to FIG. 4, fall risk scores may be determined for a plurality of data points of body movement data acquired via an IMU of a wearable fall risk monitoring device and biosensor data acquired via one or more biosensors of the wearable fall risk monitoring device. The fall risk score data may be timestamped, such that each fall risk score that is determined is associated with a particular time stamp.

At 704, method 700 includes geolocating the points of the fall risk score data. For example, as each fall risk score, which corresponds to points or windows of body movement data and biosensor data, is time stamped, the location of the wearer of the wearable device may be determined at that particular time stamp. For example, the wearable device and/or a remote device that is communicatively coupled to the wearable device may comprise a GPS, Wi-Fi, Bluetooth™, ultrawide band, RFiD, and/or other location-determining techniques that may acquire location data of the wearable device. The location data may then be time aligned with the points of the fall risk score data to identify the location of the wearable device at the time that the sensor data to which each fall risk score corresponds was acquired. Geolocating the points may include geolocating fall risk exposure events, which may be identified via fall risk score above a threshold (e.g., based on abnormal body movement data and/or biosensor data as described with respect to FIG. 4), as well as typical or baseline data.

In some examples, fall risk score data may be acquired from a plurality of wearable devices worn by a plurality of wearers. Geolocation of points of the fall risk score data may thus include geolocation of multiple wearable devices. This may allow for a greater population of fall risk scores, thereby increasing the robustness and accuracy of a generated map as well as allowing for determination of a chance fall risk event vs likely true hazards, for example based on density of events at particular locations as will be described further below.

At 706, method 700 optionally includes receiving user inputs identifying events corresponding to high fall risk scores. For example, when a wearer that is wearing a fall risk monitoring device experiences a fall risk exposure event, they may indicate on their wearable device, or on a remote device communicatively coupled to the wearable device, that there was a fall risk exposure hazard present and/or upload a photo of the hazard. For example, an application may be accessible via a remote device, and via the remote device, the user may select an element for logging a fall risk exposure event (or confirming that a fall risk exposure event occurred when the system provides a prompt in response to detection of abnormal body movement and biosensor data). Logging a fall risk exposure event may include a log in memory that a user reported event occurred, a user inputted description of the event and/or the associated hazard (e.g., “uneven sidewalk”, “icy ground”, etc.), and/or a photo of the hazard. Photos that are uploaded may also be geolocated. Thus, the user input may be associated with the body movement and biosensor data and the geolocation of the corresponding fall risk score.

At 708, method 700 includes determining a density of fall risk scores within a grid area of the map. For example, the map in which the geolocation is performed may be partitioned into a plurality of equally sized squares, thereby forming a plurality of grid areas. In some examples, determining the density of fall of fall risk scores within a grid area of the plurality of grid areas may include applying a quartic kernel function, as noted at 710, for each fall risk score. The quartic kernel function is given in equation (2):

$$\\ \begin{matrix}K ( d ) = \frac{15}{16} {( 1 - {d}^{2} )}^{2} * I & ( 2 )\end{matrix}$$

Where d is a quotient of bandwidth H and a distance D from each grid center; and I is the fall risk score. The quartic kernel function may output the density of fall risk scores within the grid area of concern.

At 712, method 700 includes identifying, from the fall risk score data, true hazard events. Determining true hazard events may comprise determining a chance distribution of fall risk scores, as noted at 712. The chance distribution may be determined according to a set of Monte Carlo simulations. The chance distribution may allow for identification of statistically significant fall risk scores, thereby likely indicating exposure to true fall hazards. For example, if an identified fall risk score falls outside the majority of that by-chance distribution, the identified fall risk score likely corresponds to a true hazard event.

At 714, method 700 includes generating a map of fall risk exposure events. In some examples, the map of fall risk exposure events may include various levels of map elements, including both density information and exposure event elements corresponding to individual fall risk exposure events (e.g., identified based on fall risk score above a predefined threshold). For example, when zoomed to a first threshold, map grid areas may be displayed with associated color coding based on density and/or fall risk scores, as noted at 716. For example, the number of true hazards within a grid area, as determined via the quartic kernel function and the chance distribution calculation, may inform the density map, whereby regions with higher density of true hazards are color coded in the map differently. As an example, a first grid area that does not have any associated true hazard events may not be colored in the map, a second grid area with low density of true hazard events may be colored yellow, and a third grid area with high density of true hazard events may be colored red. It should be understood however that other ways of designating density of events may be used, such as shading, number system, and the like.

When zoomed to a second threshold wherein each grid area takes up a larger percentage of the shown portion of the map than the first threshold, rather than displaying color coded grid areas the map may include exposure event elements, each corresponding to an identified fall risk exposure event. Each exposure event element may be displayed within the map according to the geolocation of the corresponding event.

When a user input identifying a fall risk event corresponding to a specific fall risk score is provided, that user input may be associated with the corresponding grid area and/or the corresponding exposure event element in the generated map. For example, when the map is displayed, as will be explained below, the exposure event elements may be selectable elements within the displayed map. User selection of one of the selectable elements may trigger display of a pop-up that provides additional information of the corresponding event(s), such as user descriptions, photos, and the like.

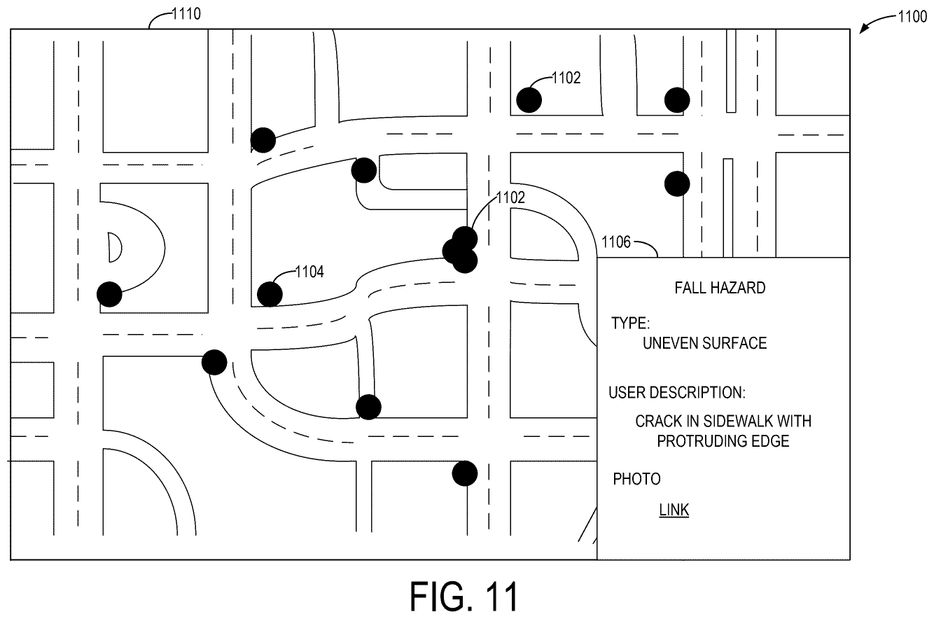

At 718, method 700 includes outputting the map of fall risk exposure events for display. For example, the map of fall risk exposure events may be displayed on a display device of the wearable fall risk monitoring device and/or a display device of a remote device (e.g., a computer, a smart phone, etc.). With the example of a smart phone, in an application associated with the wearable device, the map may be accessible via a dedicated user interface, as will be further described with respect to FIG. 11.

For example, a user may open the application within their smart phone, access the dedicated user interface of the map of fall risk exposure events, and select one or more grid areas and/or exposure event elements displayed within the map. Selection of, for example, an exposure event element, may launch a pop-up window displayed over the dedicated user interface that shows additional information about the exposure event, including any user inputs associated with the events (e.g., descriptions of the hazard, photos of the hazard, etc.). Thus, users may preview hazards as they move about an environment. Additionally, the maps may be used by industrial teams who put their efforts towards building, road, parking lot, and sidewalk maintenance, thereby allowing easy indication of areas with high amounts of fall risk exposure hazards.

In yet further examples, the map may be used to notify a wearer of upcoming exposure hazards. For example, the wearer may enter a destination that they are walking to for which a route is generated, for example by a navigation application of their mobile device. The route may then be cross-referenced with the map of fall risk exposure events to identify areas that the wearer may encounter on their trip. When an upcoming exposure hazard is identified, for example based on a high density of exposure events, the wearer may be notified that there is a potential hazard upcoming, thereby allowing the wearer to be prepared and/or to avoid walking past that particular hazard. In this way, the generated map may aid in reducing both falls in the moment as well as prevent exposure to hazards.

Turning now to FIG. 11, a generated map 1100 of fall risk exposure events is shown. The map 1100 may be displayed within a user interface 1110 on a display device, such as a watch face screen, in an application of a mobile device, in a web browser of a computer, or the like. The map 1100 is depicted zoomed in to show individual exposure events rather than grid area densities, though it should be understood that density of events within various grid areas may alternatively and/or additionally be shown in a generated map. The map 1100 includes a plurality of exposure event elements 1102. Each of the exposure event elements 1102 is selectable via user input (e.g., a mouse click, a touch to a touch screen, or the like). Upon user selection, a pop-up window may be displayed over the map 1100, for example in a side panel located at a left or a right of the display screen. In some examples, the pop-up window may be movable by the user, for example the user may hold a cursor click over the pop-up window while moving the window to a different position within the display screen.

As an example, exposure event element 1104, when selected, may launch pop-up panel 1106. The pop-up panel 1106 may include additional information related to the exposure event element 1104, including the type of fall hazard (e.g., an uneven surface, an icy surface, a slippery surface, etc.), a user description of the fall hazard (e.g., “crack in sidewalk with protruding edge”, “patch of ice on sidewalk”, or “slippery/wet region”), and/or a photo or a link to a photo of the fall hazard. The pop-up panel 1106 is shown displayed over a bottom-right corner of the user interface 1110.

As described above, the map 1100 may be generated based on body movement and biosensor data, position data, user inputs, and/or fall risk score data from a plurality of wearable devices, if the wearer has elected to share fall risk exposure data with the general population. In some examples, specific exposure events may be linked to the wearer from which they were identified, though wearer identification may or may not be displayed to other users via a corresponding pop-up panel. In other examples, exposure events may be entirely anonymous.

The technical effect of the fall risk assessment system described herein is that the system may provide a personalized and scalable solution for preventing falls in people's daily lives. The personalized assessment may help individuals recognize their fall risk, be prepared when fall hazards are nearby, and encourage them to adjust their behaviors toward safety and health. Furthermore, the data acquired and the assessment outputted by the fall risk assessment system can be applied to a large population through integration with daily use of wearable devices, such as smart-watches, rings, and the like, without additional sensing devices. As an example, when combined with GPS data of a smart watch, high-risk areas where people have a common increase in fall risk may be identified, allowing for eliminating environmental risk factors and improving public safety.

Further, integrating both body movement data and biosensor data may increase accuracy of detection of fall risk exposure events. For example, body movement data alone may result in over detection of fall risks, wherein abnormal body movement resulting from non-walking movement of a body part on which the wearable device is worn (such as pointing, gesturing, grabbing items, bending down, etc.) may erroneously be interpreted as fall risk exposures. By combining body movement data with biosensor data, accuracy of detection may be increased. For example, abnormal body movement paired with abnormal biosensor data may have a higher fall risk score than abnormal body movement alone, thereby allowing for stratification of fall risk scores and identification of the most likely fall risk exposure events.

Further, in contrast with an alternative fall prevention system that relies on a different ML model trained using supervised learning, the fall risk assessment model disclosed herein may more accurately detect abnormal behaviors, due to a difficulty in acquiring sufficient training data for the supervised learning. To achieve a desired degree of accuracy, the ML model of the alternative fall prevention system would have to be trained to compare the abnormal pattern of movements to a variety of different stored abnormal patterns corresponding to different fall scenarios. In other words, the alternative ML model may be trained to determine whether the abnormal pattern of movements corresponds to bracing behaviors observed in the training data prior to a fall; to misstep behaviors observed in the training data prior to a fall; to a sudden change of direction of the wearer observed in the training data prior to a fall; and to unexpected changes in a variety of different types of surfaces on which the wearer may be walking observed in the training data prior to a fall. Because of the high degree of variability in conditions and scenarios in which the wearer may be walking, as well as individual differences in walking patterns, the alternative ML model may perform at a desired standard in a limited number of scenarios for which adequate data is available. Therefore, the unsupervised technique disclosed herein may improve the generalizability of its use and reduce risks associated with training data bias of supervised models.

The disclosure also provides support for a method, comprising: acquiring body movement data with one or more motion sensors of a wearable fall risk monitoring device, acquiring biosensor data with one or more biosensors of the wearable fall risk monitoring device, determining, based on the body movement data and the biosensor data, a fall risk score indicating a level of fall hazard exposure, and in response to the fall risk score exceeding a threshold, notifying the wearer via one or more of the wearable fall risk monitoring device and a remote device communicatively coupled to the wearable fall risk monitoring device. In a first example of the method, the one or more motion sensors are included in an inertial measurement unit (IMU). In a second example of the method, optionally including the first example, the one or more motion sensors of the IMU comprise at least one accelerometer and at least one gyroscope. In a third example of the method, optionally including one or both of the first and second examples, the one or more biosensors comprise an electrodermal activity (EDA) sensor. In a fourth example of the method, optionally including one or more or each of the first through third examples, determining, based on the body movement data and the biosensor data, the fall risk score comprises: analyzing the body movement data to determine one or more instances of abnormal movement, analyzing the biosensor data to determine one or more instances of anomalous physiological arousal, evaluating the movement and biosensor data in combination for the one or more instances of abnormal movement and the one or more instances of anomalous physiological arousal. In a fifth example of the method, optionally including one or more or each of the first through fourth examples, the body movement data and the biosensor data are acquired as time series data and combined evaluation comprises time aligning the body movement data and the biosensor data. In a sixth example of the method, optionally including one or more or each of the first through fifth examples, analyzing the body movement data to determine one or more instances of abnormal movement comprises deploying a machine learning (ML) model that is trained using unsupervised learning. In a seventh example of the method, optionally including one or more or each of the first through sixth examples, analyzing the biosensor data to determine one or more instances of anomalous physiological arousal comprises decomposing the biosensor data to isolate high-frequency signals and summing amplitude of the high-frequency signals over a time window to determine a level of physiological response. In a eighth example of the method, optionally including one or more or each of the first through seventh examples, notifying the wearer via one or more of the wearable fall risk monitoring devices and the remote device(s) communicatively coupled to the wearable fall risk monitoring device comprises at least one of: outputting a notification to a display screen of one of the wearable fall risk monitoring device and the remote device, outputting an audio signal by one of the wearable fall risk monitoring devices and the remote device(s), and outputting a haptic feedback notification via a haptic feedback system of the wearable fall risk monitoring device.

The disclosure also provides support for a fall risk assessment system, comprising: a wearable fall risk monitoring device comprising an inertial measurement unit (IMU) and a biosensor, a microcomputer coupled to the wearable fall risk monitoring device, wherein the microcomputer is communicatively coupled to the IMU, the biosensor, and to one or more remote devices and comprises a processor and memory storing instructions executable by the processor that, when executed, cause the processor to: simultaneously acquire body movement data with the IMU and biosensor data with the biosensor, determine, for a time window, a fall risk score based on the body movement data and the biosensor data, in response to the fall risk score exceeding a threshold, outputting a notification to a wearer of the wearable fall risk monitoring device via one of the wearable fall risk monitoring device and the one or more remote devices. In a first example of the system, to determine, for the time window, the fall risk score based on the body movement data and the biosensor data, the processor is further configured to: analyze the body movement data via a machine learning (ML) model trained using unsupervised learning to detect abnormal patterns in the body movement data, and analyze the biosensor data to detect anomalous physiological arousal, and evaluate the combined analyzed body movement data and analyzed biosensor data. In a second example of the system, optionally including the first example, analyzing the biosensor data comprises applying a convex optimization-based decomposition and summing an amplitude over the time window to determine a level of response, wherein when the level of response is above a predefined threshold, anomalous physiological arousal is detected for the time window. In a third example of the system, optionally including one or both of the first and second examples, the biosensor is an electrodermal activity (EDA) sensor. In a fourth example of the system, optionally including one or more or each of the first through third examples, the body movement data is acquired while the wearer is walking, running, exercising, or dancing. In a fifth example of the system, optionally including one or more or each of the first through fourth examples, the fall risk score is a value between 0 and 1.

The disclosure also provides support for a method, comprising: acquiring a plurality of fall risk scores determined from sensor data acquired with a plurality of wearable fall risk monitoring devices, wherein each of the plurality of wearable fall risk monitoring devices comprises an inertial measurement unit (IMU) and a biosensor, wherein the sensor data of each of the plurality of wearable fall risk monitoring devices comprises body movement data acquired with the IMU and biosensor data acquired with the biosensor, and wherein the plurality of fall risk scores are determined based on the sensor data, generating, based on the plurality of fall risk scores, a map of fall risk exposure events, and outputting the map of fall risk exposure events via a display device. In a first example of the method, generating the map of fall risk exposure events comprises: geolocating the plurality of wearable fall risk monitoring devices for times when the sensor data to which the plurality of fall risk scores correspond was acquired, determining density of fall risk scores within grid areas of the map, and identifying fall risk scores of the plurality of fall risk scores that indicate true hazards. In a second example of the method, optionally including the first example, determining the density of fall risk scores within grid areas of the map comprises applying a quartic kernel function to the geolocated fall risk score data. In a third example of the method, optionally including one or both of the first and second examples, the map of fall risk exposure events comprises density of fall risk scores for grid areas of the map and one or more exposure elements corresponding to individual fall risk exposure events identified based on the plurality of fall risk scores. In a fourth example of the method, optionally including one or more or each of the first through third examples, outputting the map of fall risk exposure events via the display device comprises outputting the map for display via one or more of a remote device and a wearable fall risk monitoring device, wherein the remote device comprises one of a mobile device, a desktop computer, and a laptop computer.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
acquiring body movement data with one or more motion sensors of a wearable fall risk monitoring device;
acquiring biosensor data with one or more biosensors of the wearable fall risk monitoring device;
determining, based on the body movement data and the biosensor data, a fall risk score indicating a level of fall hazard exposure; and
in response to the fall risk score exceeding a threshold, notifying the wearer via one or more of the wearable fall risk monitoring device and a remote device communicatively coupled to the wearable fall risk monitoring device.

2. The method of claim 1, wherein the one or more motion sensors are included in an inertial measurement unit (IMU).

3. The method of claim 2, wherein the one or more motion sensors of the IMU comprise at least one accelerometer and at least one gyroscope.

4. The method of claim 1, wherein the one or more biosensors comprise an electrodermal activity (EDA) sensor.

5. The method of claim 1, wherein determining, based on the body movement data and the biosensor data, the fall risk score comprises:
analyzing the body movement data to determine one or more instances of abnormal movement;
analyzing the biosensor data to determine one or more instances of anomalous physiological arousal;
evaluating the movement and biosensor data in combination for the one or more instances of abnormal movement and the one or more instances of anomalous physiological arousal.

6. The method of claim 5, wherein the body movement data and the biosensor data are acquired as time series data and combined evaluation comprises time aligning the body movement data and the biosensor data.

7. The method of claim 5, wherein analyzing the body movement data to determine one or more instances of abnormal movement comprises deploying a machine learning (ML) model that is trained using unsupervised learning.

8. The method of claim 5, wherein analyzing the biosensor data to determine one or more instances of anomalous physiological arousal comprises decomposing the biosensor data to isolate high-frequency signals and summing amplitude of the high-frequency signals over a time window to determine a level of physiological response.

9. The method of claim 1, wherein notifying the wearer via one or more of the wearable fall risk monitoring devices and the remote device(s) communicatively coupled to the wearable fall risk monitoring device comprises at least one of:
outputting a notification to a display screen of one of the wearable fall risk monitoring device and the remote device;
outputting an audio signal by one of the wearable fall risk monitoring devices and the remote device(s); and
outputting a haptic feedback notification via a haptic feedback system of the wearable fall risk monitoring device.

10. A fall risk assessment system, comprising:
a wearable fall risk monitoring device comprising an inertial measurement unit (IMU) and a biosensor;
a microcomputer coupled to the wearable fall risk monitoring device, wherein the microcomputer is communicatively coupled to the IMU, the biosensor, and to one or more remote devices and comprises a processor and memory storing instructions executable by the processor that, when executed, cause the processor to:
simultaneously acquire body movement data with the IMU and biosensor data with the biosensor;
determine, for a time window, a fall risk score based on the body movement data and the biosensor data;
in response to the fall risk score exceeding a threshold, outputting a notification to a wearer of the wearable fall risk monitoring device via one of the wearable fall risk monitoring device and the one or more remote devices.

11. The fall risk assessment system of claim 10, wherein to determine, for the time window, the fall risk score based on the body movement data and the biosensor data, the processor is further configured to:
analyze the body movement data via a machine learning (ML) model trained using unsupervised learning to detect abnormal patterns in the body movement data; and
analyze the biosensor data to detect anomalous physiological arousal; and
evaluate the combined analyzed body movement data and analyzed biosensor data.

12. The fall risk assessment system of claim 11, wherein analyzing the biosensor data comprises applying a convex optimization-based decomposition and summing an amplitude over the time window to determine a level of response, wherein when the level of response is above a predefined threshold, anomalous physiological arousal is detected for the time window.

13. The fall risk assessment system of claim 10, wherein the biosensor is an electrodermal activity (EDA) sensor.

14. The fall risk assessment system of claim 10, wherein the body movement data is acquired while the wearer is walking, running, exercising, or dancing.

15. The fall risk assessment system of claim 10, wherein the fall risk score is a value between 0 and 1.

16. A method, comprising:
acquiring a plurality of fall risk scores determined from sensor data acquired with a plurality of wearable fall risk monitoring devices, wherein each of the plurality of wearable fall risk monitoring devices comprises an inertial measurement unit (IMU) and a biosensor, wherein the sensor data of each of the plurality of wearable fall risk monitoring devices comprises body movement data acquired with the IMU and biosensor data acquired with the biosensor, and wherein the plurality of fall risk scores are determined based on the sensor data;
generating, based on the plurality of fall risk scores, a map of fall risk exposure events; and
outputting the map of fall risk exposure events via a display device.

17. The method of claim 16, wherein generating the map of fall risk exposure events comprises:
geolocating the plurality of wearable fall risk monitoring devices for times when the sensor data to which the plurality of fall risk scores correspond was acquired;
determining density of fall risk scores within grid areas of the map; and
identifying fall risk scores of the plurality of fall risk scores that indicate true hazards.

18. The method of claim 17, wherein determining the density of fall risk scores within grid areas of the map comprises applying a quartic kernel function to the geolocated fall risk score data.

19. The method of claim 16, wherein the map of fall risk exposure events comprises density of fall risk scores for grid areas of the map and one or more exposure elements corresponding to individual fall risk exposure events identified based on the plurality of fall risk scores.

20. The method of claim 16, wherein outputting the map of fall risk exposure events via the display device comprises outputting the map for display via one or more of a remote device and a wearable fall risk monitoring device, wherein the remote device comprises one of a mobile device, a desktop computer, and a laptop computer.
